(19) United States
(12) Patent Application Publication
(10) Pub. No.: US 2005/0261775 A1
(43) Pub. Date: Nov. 24, 2005

(54) Glenoid anchor

(75) Inventors: Ines Baum, Konstanz (DE); Markus Rauscher, Seuzach (CH); Peter Wendt, Wiesendangen (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Zimmer GmbH, Winterthur (CH)

(21) Appl. No.: 11/122,132

(22) Filed: May 3, 2005

(30) Foreign Application Priority Data
May 19, 2004 (EP) 04011960.4

(51) Int. Cl. A61F 2/40

(52) U.S. Cl. 623/19.12; 623/19.13

(57) ABSTRACT

The invention relates to a glenoid anchor for a joint part having a plate which can be mounted on the resected glenoid bone and which has bores for fastening elements for the anchorage in the glenoid bone and a stump for the fastening of the joint part, with the stump being firmly connected to the plate and having a mount with a conical surface at its inner side for a press fit with a conical spigot of the joint part. The invention furthermore relates to a joint connection having a glenoid anchor and a joint part which can be fastened to the glenoid anchor.

The invention relates to a glenoid anchor for a joint part having a plate which can be mounted on the resected glenoid bone and which has bores for fastening elements for the anchorage in the glenoid bone and to a stump for the fastening of the joint part.

Artificial shoulder joints are used, for example, in arthritis with advanced abrasion of the joint surfaces of the shoulder joint or in complex humeral head fractures. With a total endoprosthesis, the joint surfaces of both the humeral head and of the joint socket of the glenoid (glenoid cavity). With a damaged rotator cuff or unsuccessful treatment with a conventional shoulder prosthesis, an inverse shoulder endoprosthesis can be used, with the artificial prosthesis head and the artificial ball socket being swapped over with respect to their positions in a natural shoulder joint.

For the fastening of the artificial ball socket or of the artificial prosthesis head to the glenoid in a conventional or inverse shoulder endoprosthesis, a separate glenoid anchor of the initially named kind is usually provided which can be anchored in the glenoid by means of bone screws. The respective joint part, ball socket or prosthesis head to be attached at the glenoid side can then in turn be mounted onto the plate and fastened to such a glenoid anchor by means of a screw connection. For this purpose, the stump of the known glenoid anchor has an internal thread and a spigot of the joint part to be attached has a corresponding external thread.

It is, however, disadvantageous in such a glenoid anchor that a premature loosening of the connection of the joint part to the glenoid anchor can occur with a screw connection. Particles or chips can in particular be abraded with a screw connection and can result in inflammation and/or can cause the aforementioned premature loosening.

It is the underlying object of the invention to provide a glenoid anchor of the initially named kind which ensures a secure anchorage in the glenoid and simultaneously a reliable connection to a joint part of a shoulder endoprosthesis.

This object is satisfied by the features of claim 1 and in particular in that the stump is firmly connected to the plate and has a mount with a conical surface on its inner side for a press fit with a conical spigot of the joint part.

The invention is in particular characterized in that a conical mount, i.e. an inner cone, is formed in the stump into which a spigot of a joint part can engage which is likewise conical in shape. A press fit can hereby be provided between the glenoid anchor and the joint part which ensures a particularly firm and stable connection, with the risk of particle abrasion and/or of premature loosening in particular being reduced.

The glenoid anchor in accordance with the invention furthermore has the advantage that the conical mount of the stump and the conical spigot of the joint part can be manufactured particularly simply. The stump and the spigot can in particular be manufactured of different materials, without there being an increased risk of particle abrasion of the softer material due to different degrees of hardness of the materials. The plate and the stump are preferably each made of metal.

Furthermore, in accordance with the invention, the conical connection between the stump and the spigot can be provided particularly easily, for example by a single blow with a defined percussive force, in particular by means of a driver, with a uniform tension being able to be reached along the total inner side of the stump with a conical connection—in contrast to a screw connection.

Advantageous embodiments of the invention are also recited in the dependent claims, in the description and in the drawing.

The mount of the glenoid anchor in accordance with the invention preferably has a whole conical angle between 3° and 5°, with the conical spigot of the joint part being able to engage into said mount with an identical conical angle while forming the press fit. A mean conical diameter between 5 and 8 mm is furthermore preferably provided. Such conical angles and conical diameters have proved to be particularly suitable with respect to the limited anchorage possibilities at the glenoid and simultaneously ensure a particularly strong hold of the press fit.

The mean wall thickness of the stump preferably amounts to between 0.3 and 2 mm. With such a mean wall thickness, which likewise takes account of the limited anchorage possibilities at the glenoid bone and has proved to be particularly suitable, the stump, which preferably projects toward the glenoid bone from the plate, can serve for the additional anchorage of the glenoid anchor in the glenoid bone since, when inserting or hammering in the spigot of the joint part, the stump diameter of the stump can be at least slightly widened to form the conical connection and an anchorage of the stump in the glenoid bone can thus be achieved.

It is furthermore proposed in accordance with the invention that the plate is arched toward the glenoid bone. The plate is preferably anchored in the resected glenoid bone in the region of the natural shoulder socket. With a plate which is arched toward the glenoid bone and which is based on the natural arch of the shoulder socket, the bone removal at the glenoid bone to be resected can be reduced. Furthermore, an arched plate which is mounted on a correspondingly shaped resected glenoid bone can be anchored more reliably to the glenoid bone due to the arch than a planar plate which is mounted onto a planar position of the glenoid bone.

In accordance with a preferred embodiment of the invention, the plate is provided with an outer surface and an inner surface for the forming of the arch, said surfaces being sections of spherical surfaces having a radius between 22 and 40 mm. The centers of the two spherical surfaces can be spaced apart from one another and the radii of the outer surface and of the inner surface can differ from one another.

It is furthermore preferred for the edges of the plate terminating in the longitudinal direction to have a spacing between 25 and 50 mm on an oval plate. An oval plate is to be understood as any plate which differs from a circular base form when neglecting the arch of the plate. This includes, for example, an ellipsoid, an egg-shaped or an elongately round plate or any other desired plate to which a longitudinal direction can be assigned. The spacing of the edges formed in the longitudinal direction of the plate defines the maximum extent of the plate.

In accordance with a particular embodiment of the invention, the stump is eccentrically attached to the plate, with the plate being able to have, for example, any aforementioned shape or any other desired shape to which a longitudinal direction can be assigned. The glenoid anchor can be matched particularly advantageously to the natural geometry of the glenoid bone and/or to the limited anchorage possibilities at the glenoid bone by the forming of the stump outside a center or outside a polar axis of the plate.

The point of intersection of the longitudinal axis of the stump with a connecting straight line of edges of the plate can preferably be displaced by an amount of 2 to 8 mm from the center of the connecting straight line to a lower edge. Furthermore, the longitudinal axis of the stump can be downwardly inclined by an angle β from 3° to 7° with respect to a perpendicular on a connecting straight line from edges of the plate. The edges are preferably each edges of an oval plate terminating in the longitudinal direction, with the longitudinal direction in particular being oriented to superior-inferior.

It is particularly advantageous for the longitudinal axis of the conical surface to coincide with the longitudinal axis of the stump. A symmetrical formation of the press fit is hereby achieved which can ensure a particularly reliable fastening of the joint part to the glenoid anchor. This in particular means that the longitudinal axis of the spigot of the joint part likewise coincides with the longitudinal axis of the stump.

In accordance with a further preferred embodiment of the invention, the stump has a plurality of longitudinal ribs, in particular blade-like longitudinal ribs, at its outer side for a rotationally secure anchorage. The longitudinal ribs extend substantially parallel to the longitudinal axis of the stump and project radially outwardly from it like fins. The longitudinal ribs preferably extend over the total height of the stump and can cut into the glenoid bone on the anchorage of the glenoid anchor into the glenoid bone in order to counter a possible rotation of the glenoid anchor.

Furthermore, in accordance with the invention, a plurality of blades, in particular ring-shaped blades, with openings for the ingrowth of bone material can project from the plate toward the glenoid bone. The ring-shaped blades are preferably attached to the plate by being pressed into bores provided just for this purpose. The strength of the anchorage of the glenoid anchor to the glenoid bone can be additionally increased by bone material grown into the openings.

The invention furthermore relates to a joint connection for a shoulder endoprosthesis having a glenoid anchor such as has been explained in the above and a joint part which can be fastened to the glenoid anchor.

For the realization of an inverse shoulder endoprosthesis, the joint part can be made in hemispherical shape and can terminate flush with an upper edge of the plate or project only slightly beyond it and can project further beyond a lower edge of the plate than beyond the upper edge, with the upper edge preferably being arranged superior and the lower edge inferior. A larger pivot region on the lower side of the joint part can hereby be made possible for a spherical bearing shell or ball socket sliding on the spherical surface of the joint part and fastenable to the humerus. An abutment of the humerus at the glenoid bone can in particular hereby be prevented.

Different hemispherical joint parts having different spherical diameters are preferably provided. The surgeon in this process can choose that joint part from a set of joint parts differing from one another which appears best suited to the respective patent with respect to the spherical diameter.

The plate can preferably be recessed in a section of the hemispherical joint part corresponding to the outer contour of the plate deviating from the circular form, and in particular being oval. The plate, which is in particular oval, can be recessed at its outer contour in the matching section of the hemispherical joint part in order to define the angular position of the plate with respect to the hemispherical joint part when the press fit takes place between the spigot of the joint part and the conical surface of the mount of the stump.

The plate and the stump can consist of a particularly biocompatible material, in particular of titanium or of a titanium alloy, and the hemispherical joint part can consist of a wear-resistant material, for example of a Cr—Mo alloy, to which Ta can additionally be mixed.

The hemispherical joint part can in particular consist of a Cr—Mo alloy and be paired with a spherical bearing shell which can be fastened to the humerus and which consists of a Cr—Mo alloy. An inverse shoulder endoprosthesis is usually made such that the hemispherical joint part and the spherical bearing shell engage exactly into one another, with—in contrast to a natural shoulder joint or a conventional shoulder endoprosthesis—no displacement of the spherical head in the socket being allowed to prevent subluxation. With an inverse shoulder endoprosthesis, two components can therefore also be paired with one another which each consist of a Cr—Mo alloy. The spherical bearing shell can, however, generally also consist of a plastic, for example of polyethylene, and the hemispherical joint part can consist of a ceramic material due to the conical connection.

For the realization of a conventional shoulder endoprosthesis, a shell-like joint part having a contour which in particular corresponds to the outer contour of the plate can be fastened to the plate. A joint head attached to the humerus can then engage into the shell-like joint part which preferably consists of a plastic, for example of polyethylene.

The invention will be described in the following by way of example with reference to the drawing. There are shown:

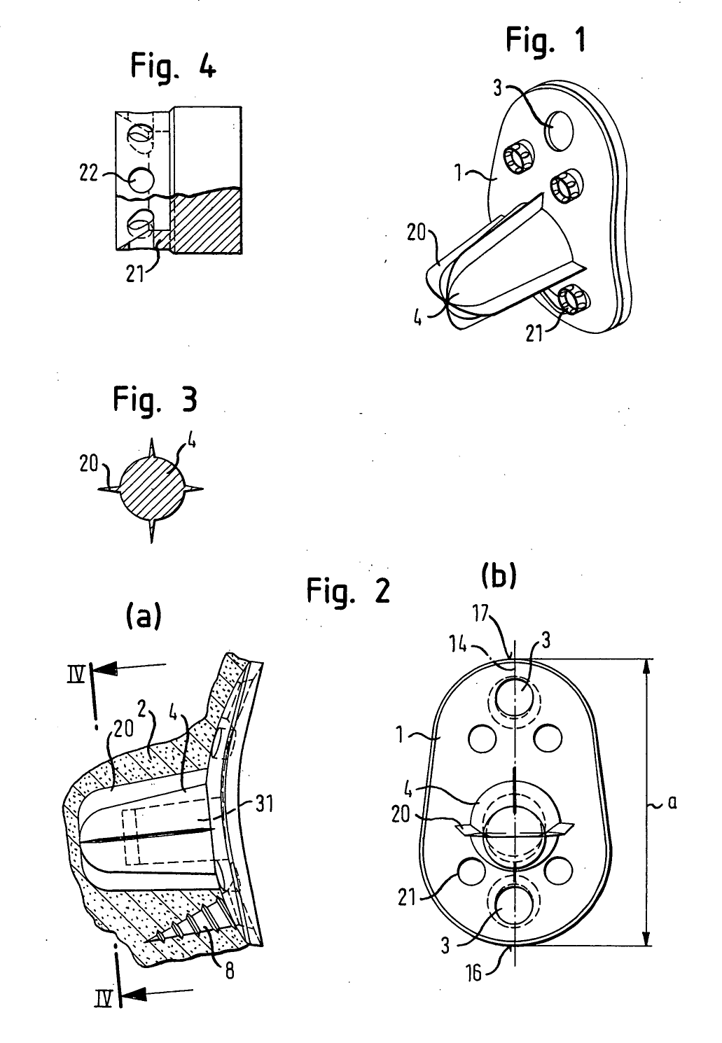

FIG. 1 a perspective view of a glenoid anchor in accordance with the invention with a plate and a stump;

FIGS. 2a, b a frontal view and a side view of the glenoid anchor of FIG. 1;

FIG. 3 a cross-section through the glenoid anchor of FIG. 2a along the line IV-IV;

FIG. 4 an enlarged illustration of a ring-shaped blade with openings attached to the plate;

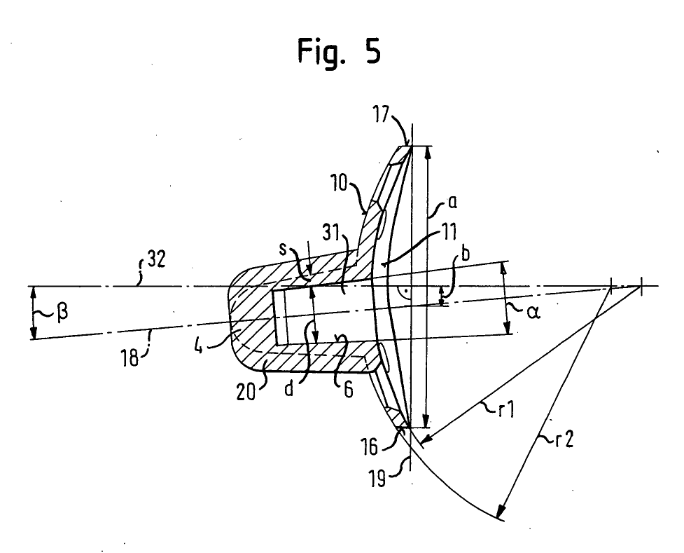

FIG. 5 a cross-section through the glenoid anchor of FIG. 2a;

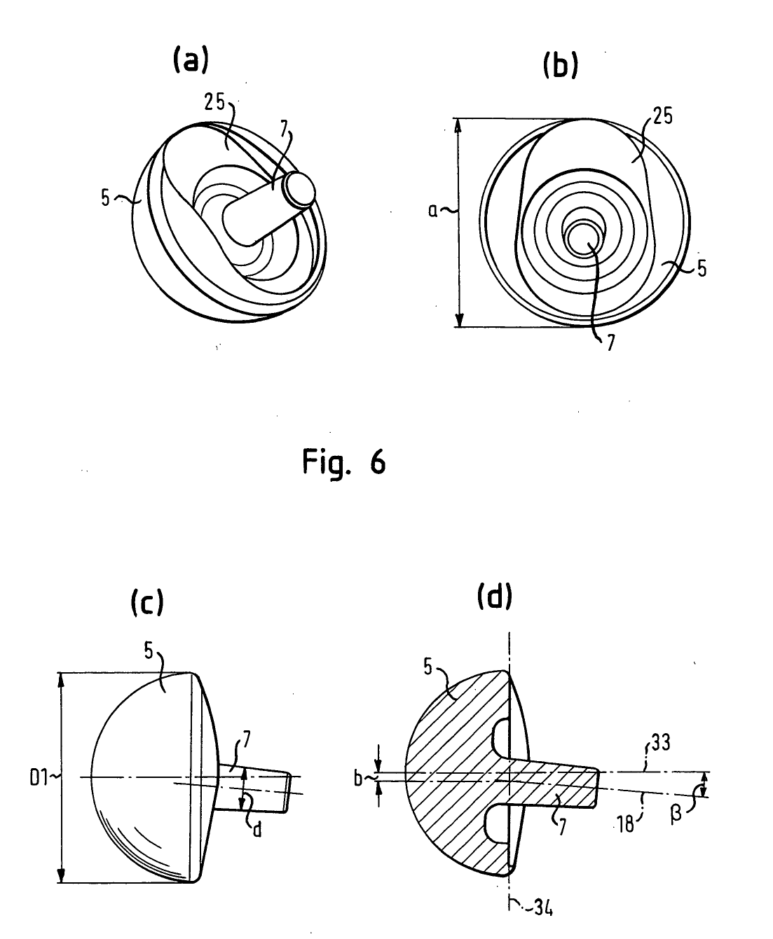

FIGS. 6a-d different views and a cross-section of a hemispherical joint part with a first ball diameter;

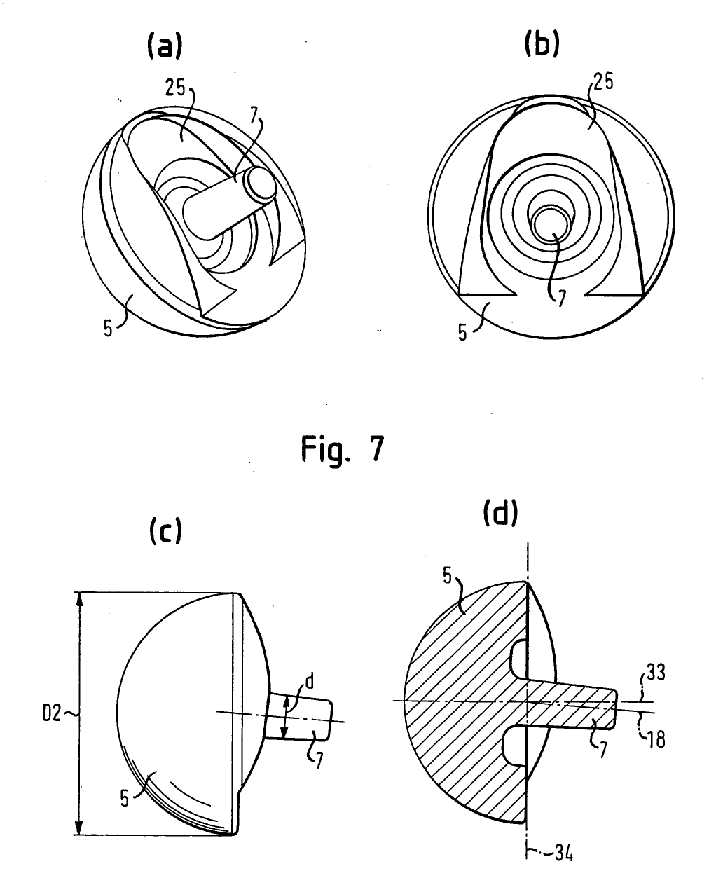

FIGS. 7a-d different views and a cross-section of a hemispherical joint part with a second ball diameter;

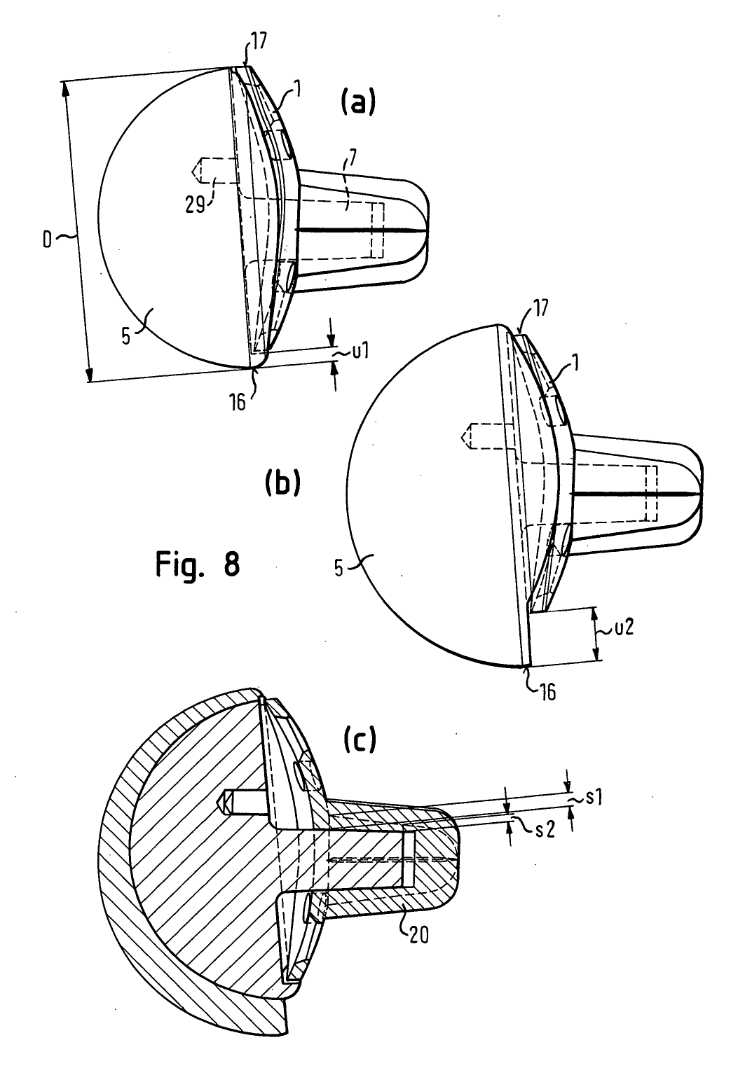

FIGS. 8a-c a joint connection in accordance with the invention with the glenoid anchor in accordance with FIGS. 1 to 5 and the hemispherical joint part in accordance with FIG. 6 or FIG. 7 in the assembled state; and

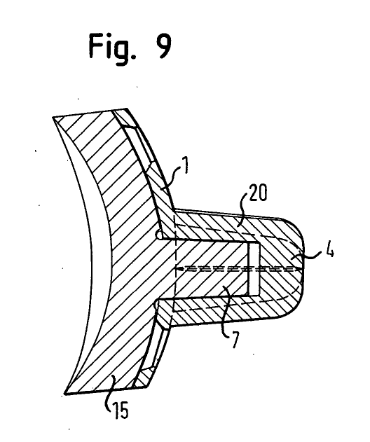

FIG. 9 a joint connection in accordance with the invention with the glenoid anchor in accordance with FIGS. 1 to 5 and a shell-like joint part in the assembled state.

FIGS. 1 to 5 show a glenoid anchor in accordance with the invention for a joint part explained in more detail at another point and having a metallic plate 1 mounted onto a resected glenoid bone 2, with the glenoid bone 2 being shown in cross-section for reasons of simplicity in FIG. 2a. Two bores 3, each for one bone screw 8, are provided in the metallic plate 1 and each serve for the anchorage of the glenoid anchor in the glenoid bone 2, with only one bone screw 8 being shown in FIG. 2a.

The plate 1, which is made of titanium or of a titanium alloy and is made with mirror symmetry to a mirror plane, has an oval base shape in the front view of FIG. 2b by which a longitudinal direction 14 of the plate 1 is defined. The two bores 3 are arranged in the region of the ends of the oval plate 1 disposed in the longitudinal direction 14 and terminated by an upper edge 17 and a lower edge 16. The edges 16, 17 terminating the plate 1 in the longitudinal direction 14 have a mutual spacing a which can lie between 25 and 50 mm.

The plate 1 is furthermore arched toward the glenoid bone 2. The plate 1 has an external surface 10, which faces the glenoid bone 2, and an internal surface 11, which is remote from the glenoid bone 2. The external surface 10 and the internal surface 11 are each designed as sections or sections of spherical surfaces to form the arching of the plate 1. The radii r1, r2 of the spherical surfaces which are associated with the external and internal surfaces 10, 11 and which can lie between 22 and 40 mm differ slightly from one another, with the centers of the two spherical surfaces being displaced with respect to one another.

A plurality of ring-shaped blades 21 are furthermore attached to the external surface 10 of the plate 1 and project from the plate 1 in the direction of the glenoid bone 2. The ring-shaped blades 21 each have a plurality of openings 22 into which the bone material grows after the anchorage of the glenoid anchor in the glenoid bone 2 in order to increase the strength of the anchorage of the glenoid anchor.

The glenoid anchor furthermore includes a thimble-like stump 4 which is firmly connected to the plate 1 and is attached to it eccentrically. The stump 4, which is made of titanium or of a titanium alloy, projects from the oval plate 1 in the direction of the glenoid bone 2 and has a plurality of blade-like longitudinal ribs 20 at its conical external surface which are substantially oriented parallel to a longitudinal axis 18 of the stump 4 and secure the glenoid anchor against rotation about the longitudinal axis 18 of the stump 4.

A connecting straight line 19 is defined between the upper edge 16 and the lower edge 17 of the plate 1. The longitudinal axis 18 of the stump 4 is downwardly inclined with respect to a perpendicular 32 on the connecting straight line 19 by an angle of inclination β which can amount to between 3° and 7°. The point of intersection of the longitudinal axis 18 of the stump with the connecting straight line 19 of the two edges 16, 17 is displaced in this process by an amount b, which can amount to 2 to 8 mm, from the center of the connecting straight line 19 in the direction of the lower edge 16 of the plate 1.

The stump 4 has a mount 31 with a conical surface 6 at its inner side. The longitudinal axis 18 of the conical surface 6 coincides with the longitudinal axis 18 of the stump 4 in this process. The cone formed in the mount 31 has a mean conical diameter d of between 5 and 8 mm and a whole conical angle or opening angle α between 3° and 5°. The mean wall thickness s of the stump 4 in the region of the conical surface 6 amounts to between 0.3 and 2 mm.

The stump 4 is designed for the securing of a hemispherical joint part 5, such as is shown in two different sizes in FIGS. 6 and 7. For this purpose, a conical spigot 7 of the joint part 5 is connected to the conical surface 6 of the mount 31 of the stump 4 in the manner of a press fit, such as is shown in FIG. 8, such that a joint connection in accordance with the invention is formed. The hemispherical joint part 5 is made from a Cr—Mo alloy and can be paired with a spherical bearing shell (not shown) which can be secured to the humerus and which is likewise made from a Cr—Mo alloy. Such a joint connection corresponds to an inverse shoulder endoprosthesis.

The mean conical diameter and the whole conical angle of the spigot 7 correspond to the conical diameter d and to the whole conical angle α of the mount 31. The longitudinal axis of the spigot 7 coincides, in the assembled state of the joint connection, with the longitudinal axis 18 of the stump 4. Analogous to the longitudinal axis of the stump 4 of the glenoid anchor, the longitudinal axis of the spigot 7 of the joint part 5 is downwardly inclined with respect to a polar axis 33 of the hemispherical joint part 5 by the angle β and its point of intersection with a base plane 34 of the hemispherical joint part 5 is offset downwardly by the amount b.

The hemispherical joint part 5 includes a section 25 whose contour corresponds to the outer contour of the oval plate 1 such that the plate 1 can be lowered in a precise fit into the section 25 of the hemispherical joint part 5 on the assembling of the joint connection. Due to the oval shape of the plate 1, whose ends disposed in the longitudinal direction 14 are not symmetrical, precisely only one angular position of the oval plate 1 can be achieved with respect to the hemispherical joint part 5.

The hemispherical joint part 5 shown in FIG. 6 having a first spherical diameter D1 terminates at the upper edge 17 of the plate 1 in a flush manner with said upper edge 17 in the assembled state of the joint connection in accordance with FIG. 8a and projects slightly beyond the lower edge 16 of the plate 1 while forming an overhang u1. The joint part 5 has an auxiliary bore 29 which serves, together with the spigot 7, for the holding of the joint part 5 on the machining of the spherical surface in the manufacture of the joint part 5.

The assembled state of a joint connection having a hemispherical joint part 5 shown in FIG. 7 with a second spherical diameter D2 which is larger than the spherical diameter D1 is shown in FIG. 8b. The joint part 5 of FIG. 7 projects slightly beyond the upper edge 17 of the plate 1 and projects further beyond the lower edge 16 of the plate 1 than beyond the upper edge 17 while forming an overhang u2. A superimposition of the assembled joint connections from FIGS. 8a and 8b is shown in FIG. 8c, with FIG. 8c in particular illustrating different wall thicknesses s1 and s2 of the stump 4.

FIG. 9 shows a further embodiment of a joint connection in accordance with the invention, with only the differences of this embodiment with respect to the embodiment shown in FIGS. 1 to 8 being explained, however. Like or corresponding parts are designated with the same reference numerals.

In contrast to FIG. 8, a joint connection is shown in FIG. 9 which has a shell-like joint part 15, instead of a hemispherical joint part, into which a joint head (not shown) attached to the humerus can engage such that a conventional shoulder endoprosthesis is formed. The shell-like joint part 15 made of polyethylene has an outer contour which corresponds to the outer contour of the oval plate 1 such that the shell-like joint part 15 does not project beyond the oval plate 1.

The anchorage of the glenoid anchor in accordance with the invention in the glenoid bone and the assembly of the joint connection in accordance with the invention are described in the following with respect to FIGS. 1 to 9.

The glenoid bone 2 is first resected using a spherical cutter such that a support surface for the plate 1 is created corresponding to the arch of the plate 1. Then a hole for the stump 4 of the glenoid anchor is drilled into the support surface by means of a central drill. The glenoid anchor can thereupon be set. For the securing of the glenoid anchor, holes are drilled for the bone screws 8 into which the bone screws 8 are finally screwed. The glenoid anchor in accordance with the invention is now firmly connected to the glenoid bone 2.

The joint part 5, 15 can then be secured to the glenoid anchor. For this purpose, the spigot 7 of the joint part 5, 15 is pressed into the mount 31 of the stump 4 and the joint part 5, 15 is oriented such that the outer contour of the plate 1 can engage into the section 25 of the joint part 5, 15. After being pressed in slightly, the joint part 5, 15 is then hammered in by means of a driver. The hinge part 5, 15 is now firmly connected to the glenoid anchor via a press fit.

With an inverse shoulder endoprosthesis, different hemispherical joint parts 5 with different spherical diameters D are available to the surgeon and the surgeon can choose the joint part 5 suitable for the patient in each case from these. The selection of the suitable joint part 5 can take place by means of trial joint parts.

The glenoid anchor in accordance with the invention includes a stump 4 which is designed both for the anchorage of the glenoid anchor in the glenoid bone 2 and for the securing of the joint part 5, 15 to the glenoid anchor, with the securing of the joint part 5, 15 being able to be realized in a particularly simple manner and simultaneously particularly reliably by means of a press fit. The stump 4 has an outer shape which has proved to be particularly suitable for a press fit for a shoulder endoprosthesis.

### REFERENCE NUMERAL LIST

1 oval plate
2 glenoid bone
3 bore
4 stump
5 hemispherical joint part
6 conical surface
7 conical spigot
8 bone screw
10 external surface
11 internal surface
14 longitudinal direction
15 shell-like joint part
16 lower edge
17 upper edge
18 longitudinal axis
19 connecting straight line
20 longitudinal rib
21 ring-shaped blade
22 opening
25 section
29 auxiliary bore
31 mount
32 perpendicular
33 polar axis
34 base plane
a spacing
b amount
d conical diameter
D spherical diameter
r radius
s wall thickness
u overhang
α opening angle
β angle of inclination

1. A glenoid anchor for a joint part (5, 15) having a plate (1) which can be mounted on the resected glenoid bone (2) and which has bores (3) for fastening elements (8) for the anchorage in the glenoid bone (2) and a stump for the fastening of the joint part (5, 15), wherein the stump (4) is firmly connected to the plate (1) and has a mount (31) with a conical surface (6) on its inner side for a press fit with a conical spigot (7) of the joint part (5, 15).

2. A glenoid anchor in accordance with claim 1, wherein the mount (31) has a mean conical diameter (d) between 5 and 8 mm.

3. A glenoid anchor in accordance with claim 1, wherein the mount (31) has a whole conical angle (α) between 3° and 5°.

4. A glenoid anchor in accordance with claim 1, wherein the mean wall thickness (s) of the stump (4) in the region of the conical surface (6) amounts to between 0.3 and 2 mm.

5. A glenoid anchor in accordance with claim 1, wherein the plate (1) is arched toward the glenoid bone (2).

6. A glenoid anchor in accordance with claim 5, wherein the plate (1) is provided with an external surface (10) and with an internal surface (11) which are recesses of spherical surfaces with a radius (r1, r2) between 22 and 40 mm.

7. A glenoid anchor in accordance with claim 1, wherein the edges (16, 17) of the plate (1) terminating in the longitudinal direction (14) have a spacing (a) between 25 and 50 mm with an oval plate (1).

8. A glenoid anchor in accordance with claim 1, wherein the stump (4) is eccentrically attached to the plate (1).

9. A glenoid anchor in accordance with claim 1, wherein the point of intersection of the longitudinal axis (18) of the stump (4) with a connecting straight line (19) of edges (16, 17) of the plate (1) is displaced by an amount (b) of 2 to 8 mm from the center of the connecting straight line (19) to a lower edge (16).

10. A glenoid anchor in accordance with claim 1, wherein the longitudinal axis (18) of the stump (4) is downwardly inclined by an angle (β) from 3° to 7° with respect to a perpendicular (32) on a connecting straight line (19) from edges (16, 17) of the plate (1).

11. A glenoid anchor in accordance with claim 1, wherein the longitudinal axis (18) of the conical surface (6) coincides with the longitudinal axis (18) of the stump (4).

12. A glenoid anchor in accordance with claim 1, wherein the stump (4) has a plurality of longitudinal ribs (20), in particular blade-like longitudinal ribs, for the rotationally secure anchorage to its outer side.

13. A glenoid anchor in accordance with claim 1, wherein a plurality of blades (21), in particular ring-shaped blades, with openings (22) for the ingrowth of bone material project from the plate (1) toward the glenoid bone (2).

14. A joint connection for a shoulder endoprosthesis having a glenoid anchor for a joint part and a joint part (5, 15) which can be secured to the glenoid anchor, said glenoid anchor having a plate, which can be mounted on the resected glenoid bone (2) and which has bores (3) for fastening elements (8) for the anchorage in the glenoid bone (2) and a stump for the fastening of the joint part (5, 15), wherein the stump (4) is firmly connected to the plate (1) and has a mount (31) with a conical surface (6) on its inner side for a press fit with a conical spigot (7) of the joint part (5, 15)

15. A joint connection in accordance with claim 14, wherein the joint part (5) is made in hemispherical shape and terminates flush with an upper edge (17) of the plate (1) or projects only slightly beyond it and projects further beyond a lower edge (16) of the plate (1) than beyond the upper edge (17).

16. A joint connection in accordance with claim 15, wherein the different hemispherical joint parts (5) are provided with different spherical diameters (D).

17. A joint connection in accordance with claim 15, wherein the plate (1) can be recessed in a recess (25) of the hemispherical joint part (5) corresponding to an outer contour of the plate (1) deviating from a circular form, and in particular being oval.

18. A joint connection in accordance with claim 15, wherein the plate (1) and the stump (4) consist of titanium or of a titanium alloy, and the hemispherical joint part (5) consists of a wear-resistant material, for example of a Cr—Mo alloy.

19. A joint connection in accordance with claim 15, wherein the hemispherical joint part (5) consists of a Cr—Mo alloy and is paired with a spherical bearing shell which can be fastened to the humerus and which consists of a Cr—Mo alloy.

20. A joint connection in accordance with claim 15, wherein the hemispherical joint part (5) consists of a Cr—Mo alloy or of ceramic material and is paired with a spherical bearing shell of plastic, for example of polyethylene, which can be fastened to the humerus.

21. A joint connection in accordance with claim 14, wherein a shell-like joint part (15) having a contour which in particular corresponds to the outer contour of the plate (1) can be fastened to the plate (1).

22. A joint connection in accordance with claim 21, wherein the shell-like joint part (15) consists of a plastic, for example of polyethylene.
